(19) United States
(12) Patent Application Publication
(10) Pub. No.: US 2014/0323562 A1
(43) Pub. Date: Oct. 30, 2014

(54) THIOSULFINATE ANTIOXIDANTS AND METHODS OF USE THEREOF

(71) Applicants: Universition of Ottawa, Ottawa (CA); GreenCentre Canada, Kingston (CA)

(72) Inventors: Philip Jessop, Kingston (CA); Paul David Thornton, Kingston (CA); Shannon Marie Decker, Burnaby (CA); Derek Pratt, Ottawa (CA); Feng Zheng, Ottawa (CA); Donna Lynn Leger, Kingston (CA)

(21) Appl. No.: 14/360,392

(22) PCT Filed: Nov. 23, 2012
(86) PCT No.: PCT/CA2012/050852

§ 371 Date: May 23, 2014

Related U.S. Application Data
(60) Provisional application No. 61/563,356, filed on Nov. 23, 2011.

(51) Int. Cl. C07C 381/00; C10L 1/24; A61K 31/198; A61Q 19/00; A61K 31/341; A61K 8/49; A61K 8/46; A61K 31/105; C07D 407/12

(52) U.S. Cl. CPC C07C 381/00; A61K 31/105; C10L 1/2431; A61K 31/198; C07D 407/12; A61K 31/341; C10L 1/2443; A61K 8/4973; A61K 8/46; A61Q 19/00
USPC 514/461; 568/22; 514/707; 514/562; 549/502; 252/406

(57) ABSTRACT

The present application provides compound of Formula X

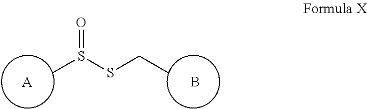

wherein A is an optionally substituted aliphatic or an optionally substituted aryl, and B is a moiety that stabilizes the partial positive charge at the carbon adjacent the divalent sulfur. The compound of Formula X is useful as a reversible antioxidant in a variety of applications. Also provided are methods of synthesizing the compound of Formula X.

### FIELD OF THE INVENTION

The present invention pertains to the field of anti-oxidants. More particularly, the present invention relates to synthetic compounds having anti-oxidant properties and methods of synthesis and use thereof.

### BACKGROUND

Garlic has been used throughout history for its medicinal properties. Studies have shown that garlic can reduce blood pressure, lower cholesterol, inhibit blood clotting, and reduce the incidence of certain cancers. It was used to disinfect open wounds during World Wars I and II and was used by Dr. Albert Schweitzer to treat cholera, typhus, and dysentery. Garlic is also known to have potent anti-fungal properties and to be helpful in treating asthma and yeast infections.

Garlic extracts from crushed garlic can contain hundreds of sulphur-containing compounds. However, allicin is often considered to be the most important of the biologically active compounds produced by crushed garlic, formed by the action of an enzyme, allinase on alliin. (Anticancer Properties of Garlic, Aug. 18, 2007, George Frederick Winter) The antioxidant effects of garlic are due to sulfenic acid, which is produced by the decomposition of allicin as shown in Scheme 1 below:

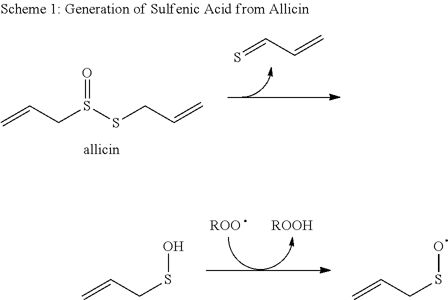

Allicin is not particularly stable and the breakdown of allicin to (E)-ajoene, as shown in Scheme 2, is not reversible. Sulfenic acid produced from the breakdown of allicin is also transient. As a result, the widespread use of allicin as an antioxidant is limited.

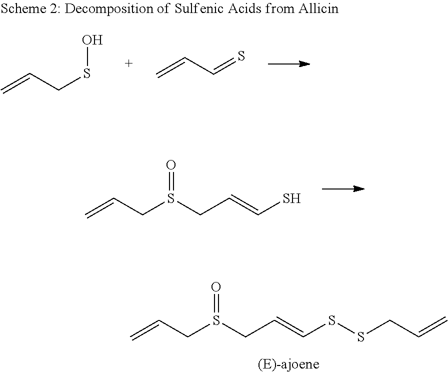

The reaction between sulfenic acid and radicals is “as fast as it can get, limited only by the time it takes for the two molecules to come into contact. No one has ever seen compounds, natural or synthetic, react this quickly as antioxidants.” (Vipraja, V., et al., Angewandte Chemie. International Edition 28 Nov. 2008)

S-Benzyl phenylmethane thiosulfinate (BPT) breaks down to form sulfenic acid by a mechanism similar to the breakdown of allicin (see Scheme 3), but, unlike allicin, BPT is stable and breaks down reversibly. In the absence of radical oxidation chain-carrying peroxyl radicals, BPT is reformed. Therefore, while allicin is an inherently unstable compound, the potential for use of BPT as a stable but highly effective antioxidant is possible. Unfortunately, the use of BPT as an antioxidant is severely restricted by its powerful odour (similar to that of garlic).

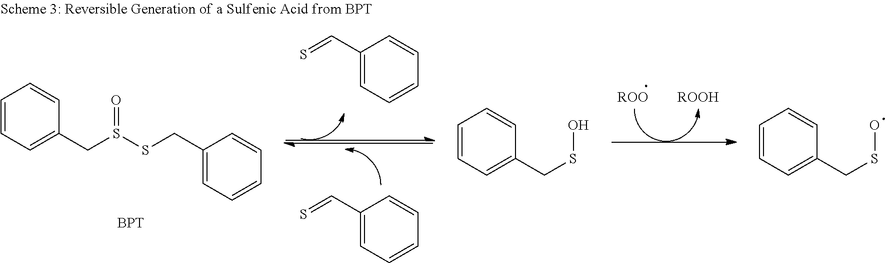

A need remains for alternative reversible antioxidants that do not have a strong odour. In certain applications it would also be beneficial for such reversible antioxidants to be more soluble in hydrocarbons than BPT, since hydrocarbons are primary targets of radical chain oxidation processes.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide reversible antioxidants and methods of use thereof

In accordance with one aspect, there is provided a compound of Formula X

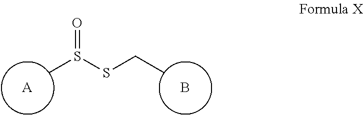

wherein
A is an optionally substituted aliphatic or an optionally substituted aryl, and
B is a moiety that stabilizes the partial positive charge at the carbon adjacent the divalent sulfur,
wherein the compound is not allicin, S-benzyl phenylmethane, petivericin, or methylpetivericin.

In one embodiment, A and B, together with the rest of the compound, form a cycle. In another embodiment, B is a substituted olefin, or an optionally substituted aryl.

In another embodiment, the compound of Formula X has the structure of Formula I

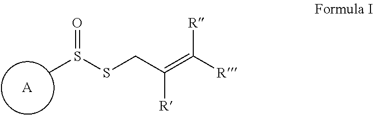

wherein
R′ is hydrogen, or optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester, and
R″ and R′″ are each independently optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester,
wherein any two or more of R′, R″ and R′″ together with the carbon atoms to which they are attached form an optionally substituted cycloalkyl or aryl ring.

In one embodiment, R′ is hydrogen, and R″ and R′″ are each independently an optionally substituted alkyl or an optionally substituted aryl.

In another embodiment, R′ and R″ together with the carbon atoms to which they are attached form an optionally substituted aryl ring, such as a phenyl, a naphthyl, an anthracenyl, a tripticenyl, a furanyl, a pyridinyl, pyrrole, thiophenyl.

In another embodiment, in the compound of Formula X, A is —CH₂-aryl, which is optionally substituted.

In one particular embodiment, the compound has the structure

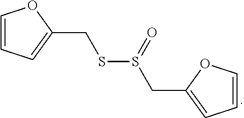 [SMILES: O=S(Cc1ccco1)SCc1ccco1]

In another embodiment, the compound has structure of Formula Ia

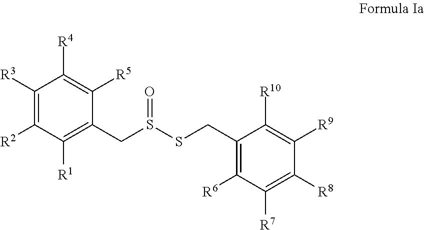

where R¹, R², R³, R⁴, R⁵, R⁶, R⁷, R⁸, R⁹ and R¹⁰ are each independently H or an optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester.
wherein at least one of R¹, R², R³, R⁴, R⁵, R⁶, R⁷, R⁸, R⁹ and R¹⁰ is not H, and
wherein when only one of R¹, R², R³, R⁴, R⁵, R⁶, R⁷, R⁸, R⁹ and R¹⁰ is not hydrogen, then one of R¹, R², R³, R⁴, R⁵, R⁶, R⁷, R⁸, R⁹ and R¹⁰ is other than methyl.

In another embodiment, at least one of R¹, R², R³, R⁴ and R⁵ is not H and at least one of R⁶, R⁷, R⁸, R⁹ and R¹⁰ is not H. In one particular embodiment, R¹, R⁵, R⁶ and R¹⁰ are H.

In another embodiment, R³ and R⁸ are each independently an optionally substituted C₄-C₂₀ alkyl. In one particular embodiment, R³ and R⁸ are the same.

In another embodiment, the compound has the structure:

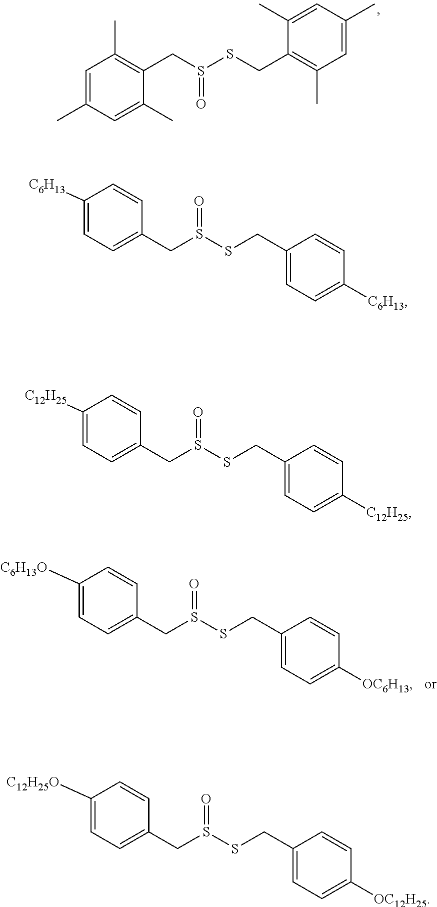

In another embodiment, there is provided a compound as described above for use as an anti-oxidant.

In another embodiment, there is provided a composition comprising a lipophilic sulfenic acid and a water soluble thiol. In one embodiment, the water soluble thiol is glutathione or cysteine.

In another aspect, there is provided a composition comprising a compound of formula X

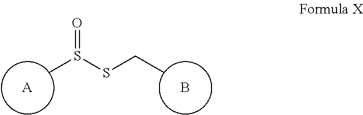

and a water soluble thiol, wherein
A is a lipophilic moiety, and
B is a moiety that stabilizes the partial positive charge at the carbon adjacent the divalent sulfur.

In one embodiment, A is a C₁₀-C₃₀ optionally substituted aliphatic or a C₁₀-C₃₀ optionally substituted aryl. In another embodiment, B is a substituted olefin, or an optionally substituted aryl. In one particular embodiment, A is —CH₂-aryl. In another embodiment, the substituted olefin of B can be of the type shown in Formula I. In another embodiment, the optionally substituted aryl of B can be of the type shown in Formula Ia.

In another aspect there is provided a method of inhibiting oxidation of a molecule comprising the step of mixing the molecule with a compound as described above.

In another aspect there is provided an antioxidant composition comprising a compound as described above and, optionally, a diluent or excipient.

In another aspect there is provided a stabilized composition comprising a compound as described above and a molecule or mixture requiring protection from radical oxidation.

In accordance with one embodiment, the antioxidant composition is mixed with a molecule, or combination of molecules, susceptible to radical oxidation, to stop or inhibit oxidation of the molecule or combination of molecules.

In another aspect, there is provided a process for preparing the compound of Formula Ia as previously defined, comprising the steps of:

a) reacting a compound of Formula II:

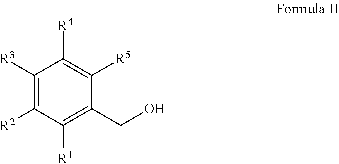

wherein R¹, R², R³, R⁴, and R⁵ are as previously defined,
with thioacetic acid and zinc iodide to form the compound of Formula III:

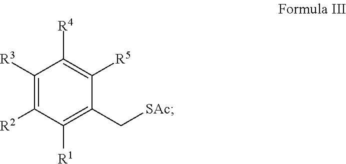

b) converting the compound of Formula III to the thiol of Formula IV:

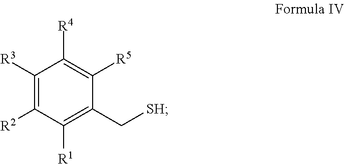

c) oxidizing the compound of Formula IV to form the disulfide of Formula V:

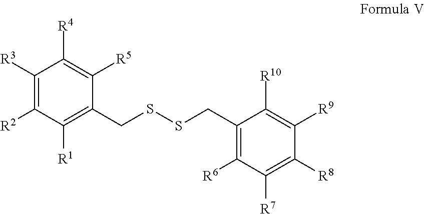

wherein R⁶, R⁷, R⁸, R⁹, and R¹⁰ are as previously defined; and
d) further oxidizing the disulfide of Formula V to form the compound of Formula Ia:

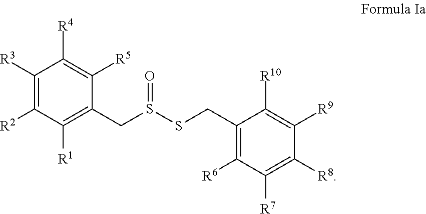

### BRIEF DESCRIPTION OF THE FIGURES

For a better understanding of the present invention, as well as other aspects and further features thereof, reference is made to the following description which is to be used in conjunction with the accompanying drawings, where:

FIG. 1 depicts representative fluorescence intensity-time profiles from MeOAMVN-mediated (0.2 mM) oxidations of egg phosphatidylcholine liposomes (1 mM) containing 0.15 μM H₂BPMHC and increasing concentrations (1.5, 3.0, 4.5, 6.0, 7.5 and 15 μM) of 1 (A), 2 (B), 5 (C) and 8 (D—15 μM profile not shown); fluorescence (λex=485 nm; λem=520 nm) was recorded every 50 s;

FIG. 2 depicts representative fluorescence intensity-time profiles from MeOAMVN-mediated (0.2 mM, left) and ABAP-mediated (2.7 mM, right) oxidations of egg phosphatidylcholine liposomes (1 mM) containing 0.15 μM H₂B-PMHC and either 2 (A, B) or 9 (C, D) at 4.5 μM (black), 7.5 μM (grey) or 15 μM (light grey) in PBS buffer of pH 7.4 containing an equivalent amount of NAC; panels E and F show the effect of NAC only (4.5, 7.5 and 15 μM); fluorescence (λex=485 nm; λem=520 nm) was recorded every 50 s; and

FIG. 3 depicts representative fluorescence-time profiles from MeOAMVN-mediated (0.2 mM, left) and ABAP-mediated (2.7 mM, right) oxidations of egg phosphatidylcholine liposomes (1 mM) containing 0.15 μM H2B-PMHC and either 4.5 μM 9 (A, B) with 1, 2, 3 or 5 equiv. of NAC or 4.5 μM 5 (C, D) with 0, 1, 2 or 4 equiv. of NAC; fluorescence (λex=485 nm; λem=520 nm) was recorded every 50 s.

### DETAILED DESCRIPTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As used herein, “aliphatic” refers to hydrocarbon moieties that are linear, branched or cyclic, may be alkyl, alkenyl or alkynyl, and may be substituted or unsubstituted. “Alkyl” refers to a linear, branched or cyclic saturated hydrocarbon group. “Alkenyl” means a hydrocarbon moiety that is linear, branched or cyclic and contains at least one carbon to carbon double bond. “Alkynyl” means a hydrocarbon moiety that is linear, branched or cyclic and contains at least one carbon to carbon triple bond. Optionally, an aliphatic moiety includes one or more heteroatoms in the linear, branched or cyclic hydrocarbon chain. For example, an aliphatic moeity can include an oxygen atom that can be within a main chain or in a substituent, in a specific example, the aliphatic moiety is an alkoxy or comprises ester.

As used herein “aryl” means a moiety including a substituted or unsubstituted aromatic ring, including heteroaryl moieties and moieties with more than one conjugated aromatic ring; optionally it may also include one or more non-aromatic ring. “C₅ to C₈ Aryl” means a moiety including a substituted or unsubstituted aromatic ring having from 5 to 8 carbon atoms in one or more conjugated aromatic rings. An aryl may have a single or multiple rings and may contain a heteroatom. Examples include, but are not limited to phenyl, naphthyl, xylenyl, phenylethanyl, anthracenyl, 1,2-dihydronaphthyl, 1,2,3,4-tetrahydronaphthyl, fluorenyl, indanyl, indenyl, pyridinyl, aziridinyl, oxiranyl, thiiranyl, azirinyl, diaziridinyl, diazirinyl, oxaziridinyl, azetidinyl, azetidinonyl, oxetanyl, thietanyl, piperidinyl, piperazinyl, morpholinyl, pyrrolyl, oxazinyl, thiazinyl, diazinyl, triazinyl, tetrazinyl, imidazolyl, benzimidazolyl, tetrazolyl, indolyl, isoquinolinyl, quinolinyl, quinazolinyl, pyrrolidinyl, purinyl, isoxazolyl, benzisoxazolyl, furanyl, furazanyl, pyridinyl, oxazolyl, benzoxazolyl, thiazolyl, benzthiazolyl, thiophenyl, pyrazolyl, triazolyl, benzodiazolyl, benzotriazolyl, pyrimidinyl, isoindolyl and indazolyl.

“Heteroaryl” means a moiety including a substituted or unsubstituted aromatic ring having from 4 to 8 carbon atoms and at least one heteroatom in one or more conjugated aromatic rings.

As used herein, “heteroatom” refers to non-carbon and non-hydrogen atoms, such as, for example, O, S, and N.

“Substituted” means having one or more substituent moieties whose presence does not interfere with the desired activity of the compound. Non-limiting examples of substituents are alkyl, alkenyl, alkynyl, aryl, aryl-halide, heteroaryl, cycloalkyl (non-aromatic ring), Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, ester, silicon-containing moieties, or a combination thereof. Preferable substituents are alkyl, aryl, heteroaryl, and ether. It is noted that aryl halides are acceptable substituents. Alkyl halides are known to be quite reactive, and are acceptable so long as they do not interfere with the desired reaction. The substituents may themselves be substituted. For instance, an amino substituent may itself be mono or independently disubstituted by further substituents defined above, such as alkyl, alkenyl, alkynyl, aryl, aryl-halide and heteroaryl cycloalkyl (non-aromatic ring).

“Short chain aliphatic” or “lower aliphatic” refers to C₁ to C₃ aliphatic. “Long chain aliphatic” or “higher aliphatic” refers to an aliphatic chain that is at least C₄ aliphatic.

As used herein, the term “lipophilic moiety” refers to a moiety is one that is capable of dissolving in fats, oils, lipids and/or non-polar solvents.

As used herein, the term “unsubstituted” refers to any open valence of an atom being occupied by hydrogen. Also, if an occupant of an open valence position on an atom is not specified then it is understood to be hydrogen.

As used herein, “olefin”, also called alkene, refers to a straight, branched or cyclic unsaturated hydrocarbon containing one or more pairs of carbon atoms linked by a double bond, and includes cyclic or acyclic olefins, in which the double bond is located between carbon atoms forming part of a cyclic (closed-ring) or of an open-chain grouping. Such olefins can be substituted or unsubstituted. Specific examples of olefins include, but are not limited to, substituted or unsubstituted 1-propene, 1-butene, 1-pentene, 1-hexene, and 1-octene and substituted or unsubstituted norbornene.

As used herein, the term “water soluble thiol” refers to a molecule having a thiol group which is moderately soluble in water.

The present application provides antioxidant compounds of Formula X

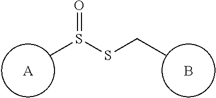

A is an optionally substituted aliphatic or an optionally substituted aryl, and
B is a moiety that stabilizes the partial positive charge at the carbon adjacent the divalent sulfur.

In some embodiments, A can be an optionally substituted aliphatic moiety, an optionally substituted aryl, an optionally substituted heteroaryl, an ester, an amide, an amine, or a silane. In other embodiments, B is an optionally substituted aryl, an optionally substituted heteroaryl or an optionally substituted bulky olefin, wherein when B is phenyl, the phenyl is substituted at at least one position.

The present application provides a compound of Formula I

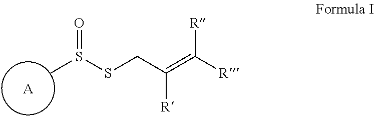

wherein
R′ is hydrogen, or optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester, and
R″ and R′″ are each independently optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester,
wherein any two or more of R′, R″ and R′″ together with the carbon atoms to which they are attached form an optionally substituted cycloalkyl or aryl ring.

The present application also provides a compound of Formula Ia

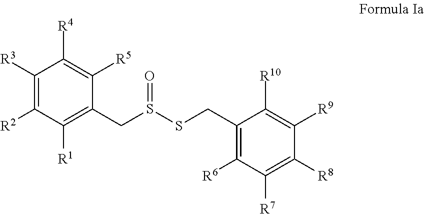

where R¹, R², R³, R⁴, R⁵, R⁶, R⁷, R⁸, R⁹ and R¹⁰ are each independently H or an optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester, wherein at least one of R¹, R², R³, R⁴, R⁵, R⁶, R⁷, R⁸, R⁹ and R¹⁰ is not H, and wherein when only one of R¹, R², R³, R⁴, R⁵, R⁶, R⁷, R⁸, R⁹ and R¹⁰ is not hydrogen, then one of R¹, R², R³, R⁴, R⁵, R⁶, R⁷, R⁸, R⁹ and R¹⁰ is other than methyl.

In one embodiment, there is provided a compound of Formula Ia wherein at least one of R¹, R², R³, R⁴ and R⁵ is not H and at least one of R⁶, R⁷, R⁸, R⁹ and R¹⁰ is not H. In a related embodiment, there is provided a compound of Formula Ia wherein R¹, R⁵, R⁶ and R¹⁰ are H. In a related embodiment, there is provided a compound of Formula Ia wherein R³ and R⁸ are each independently an optionally substituted C₄-C₂₀ alkyl. In one example, R³ and R⁸ are the same.

The compound of Formula Ia is an analogue of the natural product S-Benzyl phenylmethane thiosulfinate (BPT), and has been found to also be a reversible antioxidant. Unlike BPT, however, the compound of Formula Ia does not have a strong odour. Further, because of the presence of at least one long chain aliphatic (i.e., at least a C₄ aliphatic), the compound of Formula Ia has a greater solubility in hydrocarbons (i.e., lipids) than BPT, which are primary targets of radical chain oxidation processes.

The present application further provides methods for synthesizing a compound of Formula Ia. In one embodiment, the method comprises the step of treating a disulfide compound of Formula II with an oxidant according to the scheme below:

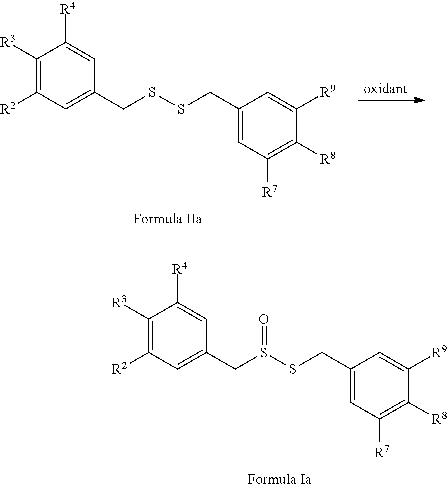

In one embodiment, the oxidant is a peroxycarboxylic acid, such as, meta-chloroperoxybenzoic acid (mCPBA). As would be appreciated by a skilled worker, the disulfide compound of Formula Ia can be prepared using a variety of synthetic methods. In one example, the disulfide is prepared according to the general method summarized in the scheme below:

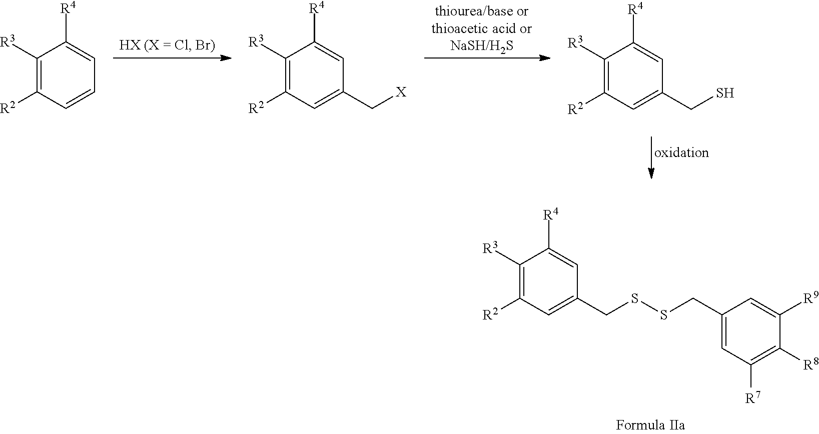

In another embodiment, the compound of Formula Ia may be prepared by the following method:

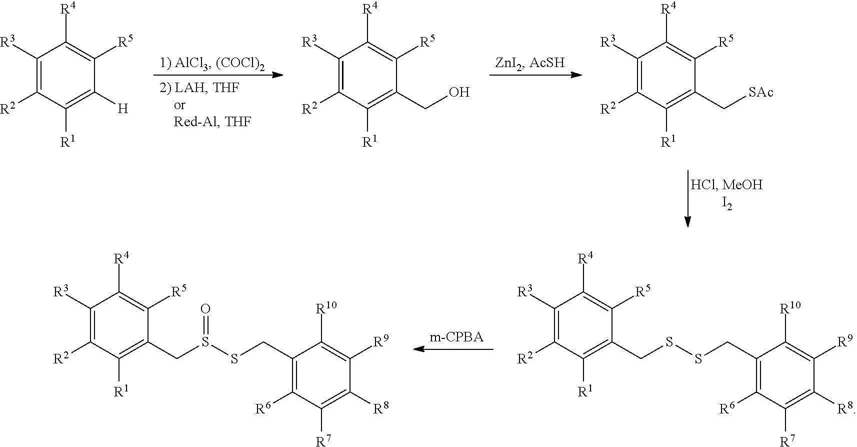

In yet another embodiment, the following method can be used to prepare the compound of Formula 1a:

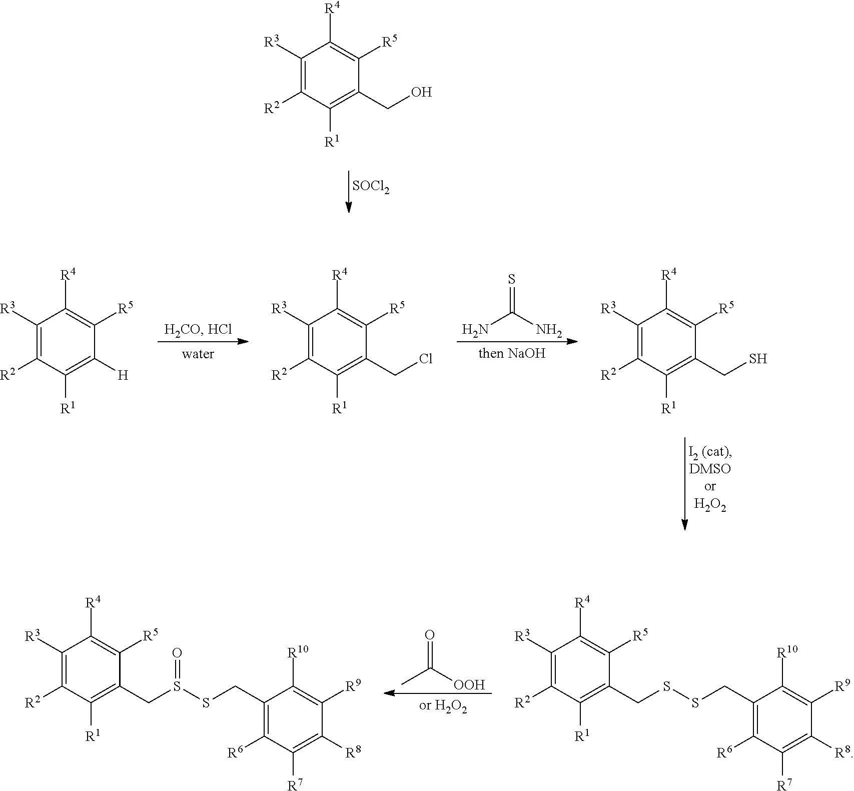

Another specific example of a compound of Formula I may be prepared as follows:

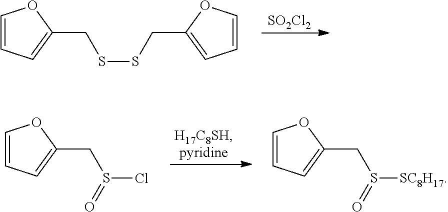

Further synthetic methods are detailed in the Examples below.

Use of Compound of Formula Ia as Exemplary of a Reversible Anti-Oxidant

Similar to BPT, and as noted above, the compound of Formula X can be a reversible anti-oxidant and will react with peroxyl radicals according to the general reaction depicted below, using a compound of Formula Ia as an exemplary compound, wherein R¹, R⁵, R⁶ and R¹⁰ are all hydrogen:

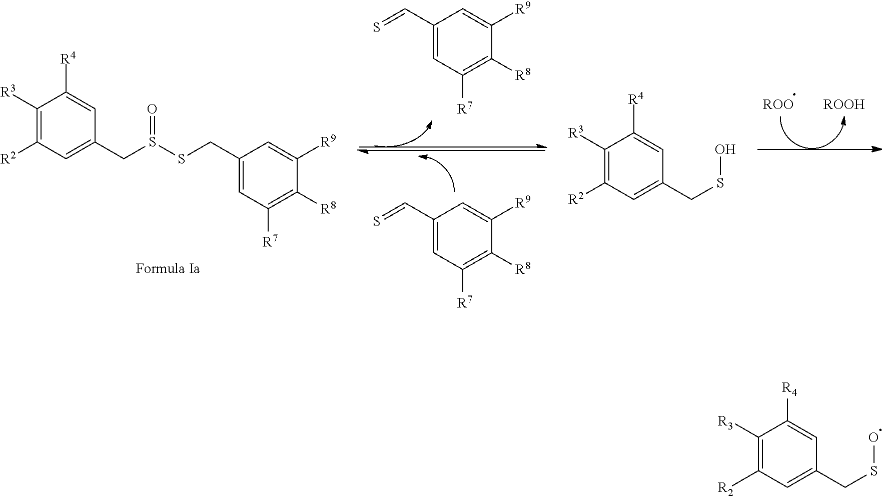

If there are no peroxyl radicals present, the compound of Formula X is reformed. As a result, the compound of Formula X can be used as a stable but highly effective antioxidant.

In one embodiment, the compound of Formula X can be used as an industrial antioxidant, for example, it can be used as a food preservative, a preservative in cosmetics, a processing aid and/or stabilizer for plastics and rubber, a polymer stabilizer, a gasoline stabilizer, a lubricant, an adhesive or in the prevention of metal corrosion. Accordingly, the present application further provides a stabilized or preserved composition comprising a compound of Formula X and a molecule or mixture to be stabilized or preserved by inhibition of radical oxidation.

Further to the application of the compound of Formula X to stabilizing gasoline, it is noted that organic sulfur compounds such as thiols and thiophenols must be removed from crude oil/fuel, due to the fact that they poison the expensive platforming catalyst and have an unpleasant odour. One of the processes used to remove these organic sulfur compounds is the MEROX sweetener process, wherein these compounds are oxidized to disulfides and returned to the fuel. The resulting disulfides can be further oxidized according to the methods noted above and known to those of skill in the art to produce thiosulfinates. It is envisaged that at least a portion of the mixture of oxidized disulfide compounds will comprise compounds of Formula X, which can then serve as antioxidants for the fuel.

In an alternative embodiment, the compound of Formula X can be used as a therapeutic anti-oxidant, for example, as a nutritional supplement or as a pharmaceutical. In this embodiment, the compound of Formula X would be formulated for administration to a subject by a route that is effective for delivering the compound and, thereby, providing the anti-oxidant effect. Suitable routes of administration include intravenous, topical, oral, intranasal, intravaginal and intrarectal. The therapeutic compounds can be administered as a composition with a pharmaceutically acceptable diluent or excipient.

The present application further provides a composition comprising the compound of Formula X, or a mixture of compounds of Formula X. The composition optionally comprises a diluent or excipient, which can be a pharmaceutically acceptable diluent or excipient.

Examples of the compounds of Formulae X and Ia include the following:

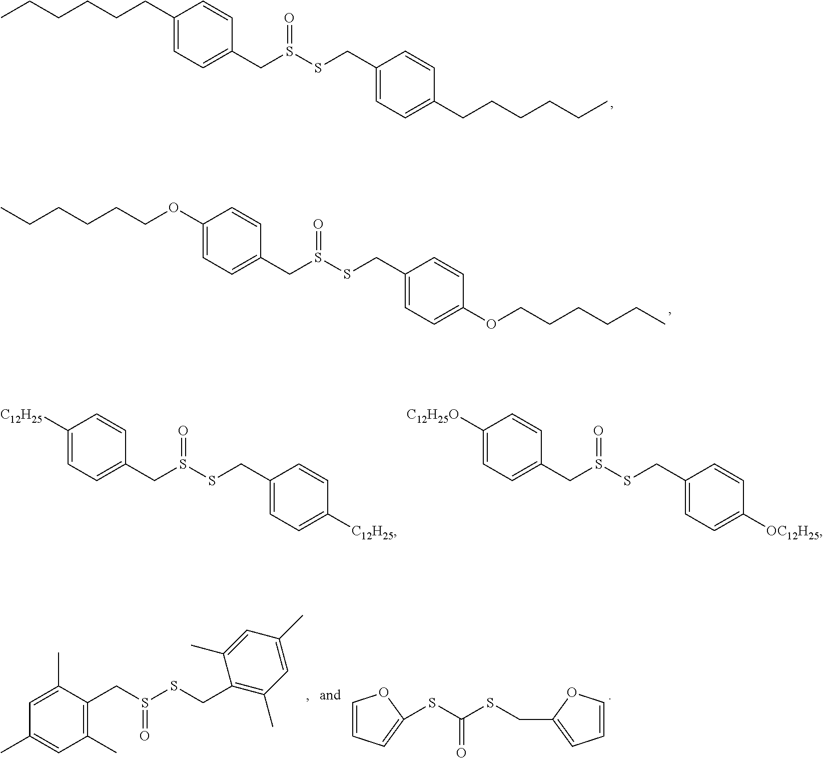

To gain a better understanding of the invention described herein, the following examples are set forth. It should be understood that these examples are for illustrative purposes only. Therefore, they should not limit the scope of this invention in any way.

### EXAMPLES

### Example 1

### Synthesis and Characterization of p-n-C₆H₁₃-Benzyl Phenylmethane Thiosulfinate

p-n-C₆H₁₃-Benzyl phenylmethane thiosulfinate was prepared according to the following scheme:

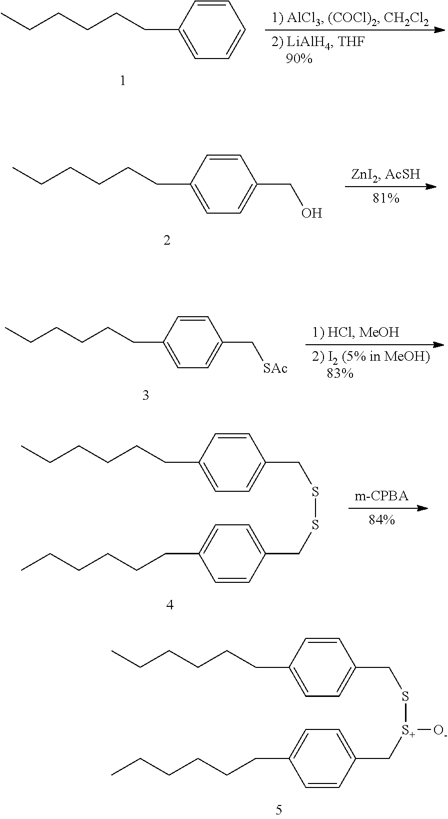

### 4-hexylphenyl)methanol 2

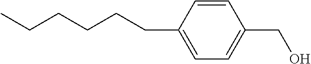

A solution of hexylbenzene (1.62 g, 10 mmol) and oxalyl chloride (1.4 g, 11 mmol) was prepared in dry DCM (15 mL). Anhydrous AlCl₃ (2.0 g, 15 mmol) was added to the solution portionly at 0° C. under argon. Anhydrous THF (10 mL) was added after the mixture was stirred at 0° C. for 1 h, and then LiAlH₄ (760 mg, 20 mmol) was added slowly and portionly. After stirring at 0° C. at 1 h, water (5 mL) was added slowly to quench the reaction. The solution was decanted from the resulting solid, which was then washed with ether. The decanted solution was combined with the ether washes and solvent was evaporated and the resultant crude product was purified by flash chromatography on silica gel (hexane:ethyl acetate=5:1) to afford compound 2 (1.73 g, 90% yield).

¹H NMR: 7.06-7.18 (AB, 4H), 4.52 (s, 2H), 2.51 (t, J=7.5 Hz, 2H), 2.18 (br, 1H), 1.52 (m, 2H), 1.16-1.29 (m, 6H), 0.80 (t, J=6.9 Hz, 3H). These spectroscopic details are in accordance with literature reports.

### S-4-hexylbenzyl ethanethioate 3

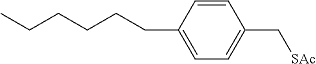

AcSH (1.37 g, 18 mmol) and ZnI₂ (954 mg, 3 mmol) were added sequentially to a solution of (4-hexylphenyl)methanol 2 (1.15 g, 6 mmol) in dichloromethane. After refluxing overnight, the reaction was quenched with water. The organic layer was separated and the water phase was extracted with ether. All the organic phases were combined and the solvent was removed under reduced pressure. The resulting oil was purified by flash chromatography to give compound 3 (1.21 g, 81% yield).

¹H NMR (300 MHz, CDCl₃): δ 6.99-7.11 (AB, 4H), 4.00 (s, 2H), 2.47 (t, J=7.5 Hz, 2H), 2.23 (s, 3H), 2.04 (m, 2H), 1.21-1.28 (m, 6H), 0.79 (t, J=6.6 Hz, 3H); ¹³C NMR (75.5 MHz, CDCl3): δ 195.0, 141.9, 134.5, 128.57, 128.52, 33.5, 33.1, 31.6, 31.3, 30.2, 28.9, 22.5, 14.0; EI (M+): 250.1.

### 1,2-bis(4-hexylbenzyl)disulfane 4

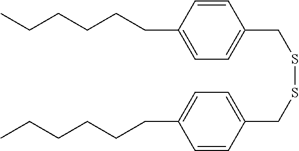 [SMILES: CCCCCCc1ccc(CSSCc2ccc(CCCCCC)cc2)cc1]

Hydrochloric acid (35-38%, 20 drops) was added to a solution of S-4-hexylbenzyl ethanethioate 3 (1.21 g, 0.48 mmol) in methanol (35 mL) at room temperature. The mixture was refluxed for 10 h and then cooled down to 0° C. Iodine solution (5% in methanol) was added dropwise until the reddish color of iodine remained. The reaction was stirred for an additional 20 min at 0° C. Sodium thiosulfate was added to kill the excess iodine. The mixture was evaporated to remove most of the methanol. The residual was treated with water (15 mL) and the resulting solution was extracted with ether. The extracts were combined, dried over magnesium sulphate and the solvent was removed under vacuum. The resulting oil was purified by flash chromatography to give compound 4 (832 mg, 83% yield).

¹H NMR (300 MHz, CDCl₃): δ 7.01-7.07 (AB, 8H), 3.49 (s, 4H), 2.49 (t, J=7.5 Hz, 4H), 1.50 (m, 4H), 1.15-1.27 (m, 12H), 0.78 (t, J=7.2 Hz, 6H); ¹³C NMR (75.5 MHz, CDCl3): δ 142.3, 134.5, 129.4, 128.6, 43.2, 35.7, 31.8, 31.5, 29.0, 22.7, 14.2; EI (M+): 414.2, (M+/2): 207.1.

### p-n-C₆H₁₃-BPT 5

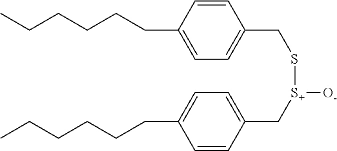

m-Chloroperbenzoic acid (m-CPBA) (77%, 517 mg, 2.31 mmol) in dichloromethane (1 mL) was added dropwise to a solution of 1,2-bis(4-hexylbenzyl)disulfane 4 (909 mg, 2.19 mmol) in dichloromethane (10 mL) at 0° C. The mixture was stirred at 0° C. for one hour. Sodium carbonate (2 g) was added in small portions with vigorous stirring. The reaction mixture was stirred for an additional 1 h at 0° C. The reaction mixture was then filtered through magnesium sulfate. The filtrate was concentrated under reduced pressure yielding crude product, which was recrystallized from ether to yield pure 5 as a white solid (575 mg, 61% yield).

¹H NMR (300 MHz, CDCl₃): δ 7.02-7.18 (m, 8H), 4.11-4.25 (m, 4H), 2.47-2.55 (m, 4H), 1.51 (m, 4H), 1.19-1.27 (m, 12H), 0.81 (m, 6H); ¹³C NMR (75.5 MHz, CDCl₃): δ 143.6, 142.6, 133.7, 128.9, 128.8, 127.1, 61.9, 35.9, 35.7, 35.6, 31.7, 31.3, 31.2, 29.0, 22.6, 14.1; EI: 190.1, 139.0.

### Example 2

### Kinetics of Reaction of p-n-C₆H₁₃-BPT with Peroxyls

The kinetics of reaction of p-n-C₆H₁₃-BPT with peroxyls was studied using a method based on the peroxyl radical clocks described in Roschek, B.; Tallman, K. A.; Rector, C. L.; Gillmore, J. G.; Pratt, D. A.; Punta, C.; Porter, N. A. J. Org. Chem. 2006, 71, 3527-3532. A comparison of the results obtained using p-n-C₆H₁₃-BPT to other antioxidants, such as BPT, was used to demonstrate the antioxidant activity of p-n-C₆H₁₃-BPT.

Stock solutions of methyl linoleate (“MeLin”) (1.0 M), methoxy 2,2′-Azobis(4-methoxy-2,4-dimethyl valeronitrile) (“MeOAMVN”) (0.1 M), and the thiosulfinates, BPT and p-n-C₆H₁₃-BPT 5, were prepared in chlorobenzene. Samples were assembled in 1 mL HPLC autosampler vials with a total reaction volume of 100 μL. Solutions were prepared in the following order to avoid premature oxidation:thiosulfinate (1 mM-7 mM), MeLin (0.10 M) and then MeOAMVN (0.01 M), and diluted to 100 μL with chlorobenzene. The sealed samples were then heated to 37° C. for 2 h. After 2 h, the oxidation was stopped by the addition of butylated hydroxytoluene (“BHT”) (50 μL of 1.0 M solution in hexanes), followed by reduction of the hydroperoxides to alcohols by triphenylphosphine (50 μL of 1 M solution in chlorobenzene). The samples were then diluted to 1 mL with HPLC grade hexanes and analyzed by HPLC (0.5% i-PrOH in hexanes, 1 mL min-1, 40 min, Sun-Fire Silica, 5 mm 4.6×250 mm column, UV detection at 234 nm). The ratio of products (Z,E:E,E) was plotted versus thiosulfinate concentration to derive k_{H}, (Roschek, B.; Tallman, K. A.; Rector, C. L.; Gillmore, J. G.; Pratt, D. A.; Punta, C.; Porter, N. A. J. Org. Chem. 2006, 71, 3527-3532).

The results of this example showed p-n-C₆H₁₃-BPT 5 to have a rate constant that is about 1.6-fold higher than that of BPT under the exact same conditions (k_{H}=2.0×10⁵ M⁻¹ s⁻¹). (Lynett, P. T.; Butts, K.; Vaidya, V.; Garrett, G. E.; Pratt, D. A. Org Biomol. Chem. 2011, 9, 3320-3330).

### Example 3

### Radical-Trapping Antioxidant Generation & Regeneration; Combination of Lipophilic Thiosulfinates and Hydrophilic Thiols

Garlic-derived thiosulfinate allicin (1), (C. J. Cavallito, J. H. Bailey, J. S. Buck, J. Am. Chem. Soc. 1945, 67, 1032-1033.) and anamu-derived petivericin (2), (R. Kubec, S. Kim, R. A. Musah, Phytochemistry 2002, 61, 675-680) are purported to be potent radical-trapping antioxidants, (Y. Okada, K. Tanaka, E. Sato, H. Okajima, Org. Biomol. Chem. 2006, 4, 4113; and Y. Okada, K. Tanaka, E. Sato, H. Okajima, Org. Biomol. Chem. 2008, 6, 1097) a reactivity to which many of the biological activities of extracts of these plants have been ascribed.

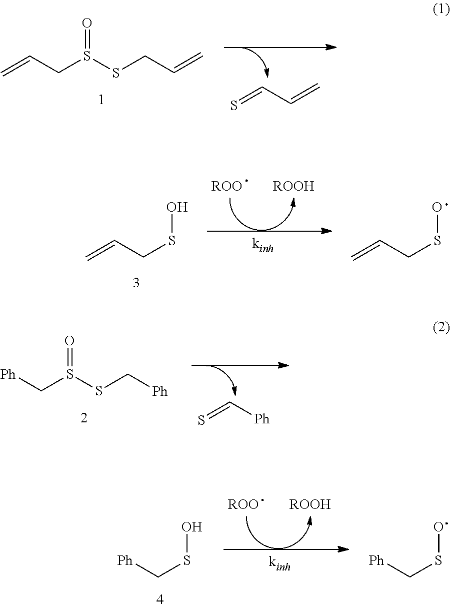

In organic solution, 1 and 2 themselves are not radical trapping antioxidants and must first undergo Cope elimination to form the sulfenic acids 3 and 4, respectively (Eq. 1 and 2), which are believed to undergo very fast reactions with peroxyl radicals (V. Vaidya, K. U. Ingold, D. A. Pratt, Angew. Chem. Int. Ed. 2009, 48, 157-160; and P. T. Lynett, K. Butts, V. Vaidya, G. E. Garrett, D. A. Pratt, Org. Biomol. Chem. 2011, 9, 3320-3330). Since the reactivities of 3 and 4 cannot be determined directly due to their short lifetimes in solution, studies of the persistent 9-triptycenesulfenic acid (5) were carried out to provide direct thermodynamic (A. J. McGrath, G. E. Garrett, L. Valgimigli, D. A. Pratt, J. Am. Chem. Soc. 2010, 132, 16759-16761) and kinetic (R. Amorati, P. T. Lynett, L. Valgimigli, D. A. Pratt, Chem. Eur. J. 2012, 18, 6370-6379) support for this hypothesis; the O—H bond strength of 5 was determined to be 72 kcal/mol, making the reaction of sulfenic acids with peroxyl radicals exothermic by ˜16 kcal/mol, and the rate constant for this reaction was determined to be kinh=3×106 M−1 s−1, making the reactions of sulfenic acids with peroxyl radicals among the fastest known. However, it remains unclear if (and how) thiosulfinates are effective radical-trapping antioxidants in biphasic media, such as biological membranes and lipoproteins, where peroxyl radicals carry the chain oxidation of polyunsaturated fatty acids implicated in the onset and development of degenerative disease (N. A. Porter, Acc. Chem. Res. 1986, 19, 262-268; and D. A. Pratt, K. A. Tallman, N. A. Porter, Acc. Chem. Res. 2011, 44, 458-467).

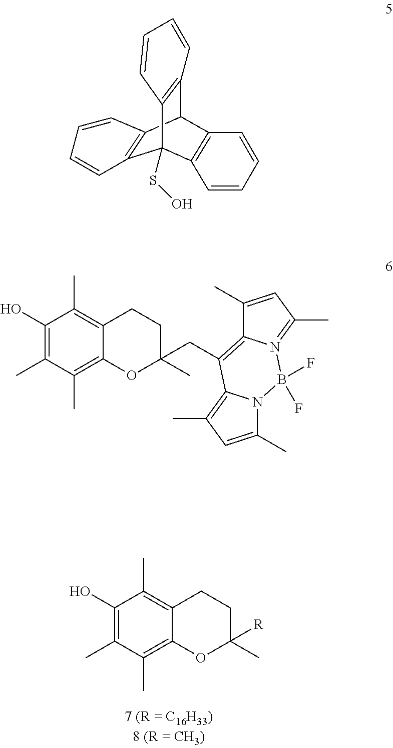

To shed some light on this issue, the radical-trapping abilities of 1, 2 and 5 have been studied in unilamellar phosphatidylcholine liposomes—convenient models for the lipid bilayers that make up biological membranes. The relative reactivities of the compounds were determined using H₂B-PMHC (6), an analog of α-tocopherol (7 or α-TOH, the most biologically-active form of Vitamin E and Nature's premier lipophilic radical-trapping antioxidant) (G. W. Burton, K. U. Ingold, Acc. Chem. Res. 1986, 19, 194-201), which undergoes a significant fluorescence enhancement upon oxidation by two equivalents of peroxyl radical. In the presence of a good radical-trapping antioxidant, the fluorescence onset is suppressed since the radicals are trapped by the compound under investigation in lieu of reacting with H₂B-PMHC.

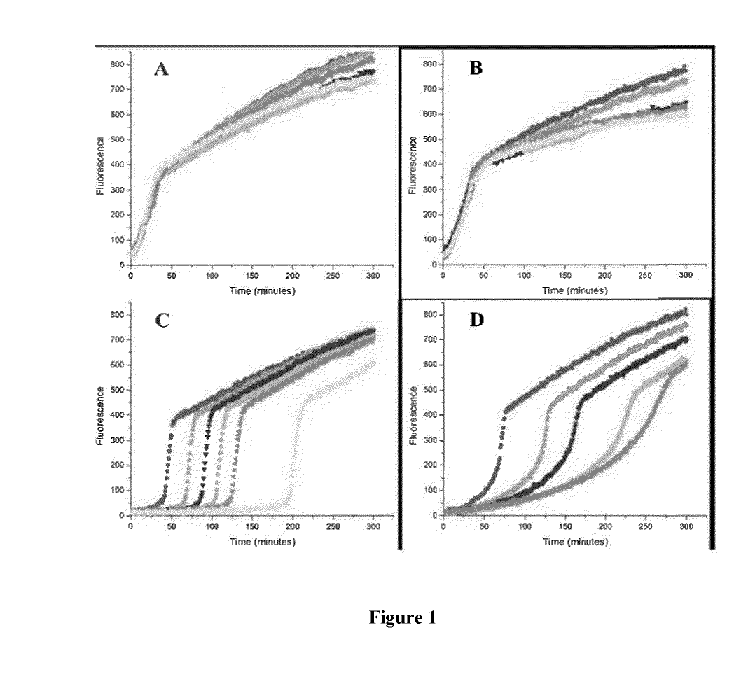

Initial experiments focused on the ability of either 1 or 2 to inhibit the oxidation of liposome-embedded H₂B-PMHC by either lipophilic or hydrophilic peroxyl radicals derived from the azo initiators MeOAMVN (N. Noguchi, H. Yamashita, N. Gotoh, Y. Yamamoto, R. Numano, E. Niki, Free Rad. Biol. Med. 1998, 24, 259-268) and ABAP, (L. R. C. Barclay, S. J. Locke, J. M. MacNeil, J. VanKessel, G. W. Burton, K. U. Ingold, J. Am. Chem. Soc. 1984, 106, 2479-2481) respectively. Representative results are shown for MeOAMVN-mediated oxidations in FIG. 1. In fact, neither 1 nor 2 were able to slow the oxidation of H₂BPMHC up to concentrations of 15 μM (100-fold higher than H₂BPMHC, FIGS. 1A and B). Since the reactivity of H₂B-PMHC is essentially the same as that of α-TOH, the efficacy of 1 and 2 must therefore be less than 1/100 that of α-TOH in a lipid bilayer. For comparison, liposome oxidations to which the persistent sulfenic acid 5 (FIG. 1C) was added were also carried out, revealing excellent concentration-dependent radical-trapping activity. In the presence of 5, the rate of H₂B-PMHC oxidation (fluorescence increase) was essentially nil, indicating a reactivity superior to that of PMHC (8, FIG. 1D), the oft used truncated model of α-TOH upon which H₂BPMHC is based. Similar results were obtained when the oxidations were carried out with water-soluble ABAP, suggesting that 5 can trap radicals at the lipid/water interface (as can 8), while allicin and petivericin cannot.

The time required to reach maximum fluorescence in the first phase of the plots in FIG. 1 (ca. 400 counts), which we will hereafter refer to as the ‘inhibited period’, reflects the stoichiometry of the reaction of the antioxidant and the peroxyl radicals. The inhibited period increases linearly with concentration of either 5 or 8, but the slope of the line for the former is roughly half of that obtained for the latter (14 min/μM for 5 versus 31 min/μM for 8 when radicals are generated using MeOAMVN and 10 min/μM for 5 versus 21 min/μM for 8 when radicals are generated using AAPH. Since it is known that 8 traps two peroxyl radicals under these conditions, (L. R. C. Barclay, S. J. Locke, J. M. MacNeil, J. VanKessel, G. W. Burton, K. U. Ingold, J. Am. Chem. Soc. 1984, 106, 2479-2481) these results imply that 5 traps only one. Indeed, in previous work in homogenous organic solutions, stoichiometric numbers ≦1 were found (V. Vaidya, K. U. Ingold, D. A. Pratt, Angew. Chem. Int. Ed. 2009, 48, 157-160; and R. Amorati, P. T. Lynett, L. Valgimigli, D. A. Pratt, Chem. Eur. J. 2012, 18, 6370-6379)

The lack of radical-trapping activity of allicin and petivericin obvious from FIGS. 1A and 1B could be due to their partitioning to the aqueous phase, where Cope elimination to produce the reactive sulfenic acids 3 and 4, respectively, is slowed dramatically by H-bonding. (V. Vaidya, K. U. Ingold, D. A. Pratt, Angew. Chem. Int. Ed. 2009, 48, 157-160; P. T. Lynett, K. Butts, V. Vaidya, G. E. Garrett, D. A. Pratt, Org. Biomol. Chem. 2011, 9, 3320-3330; E. Block, Angew. Chem. Int. Ed. Engl. 1992, 31, 1135-1178; and F. Freeman, Y. Kodera, J. Agric. Food Chem. 1995, 43, 2332-2338] However, the half-life for decomposition of allicin in the liposome solutions (determined by HPLC) was extended by only 50% compared to that determined in non-H-bonding organic solvents (˜60 minutes). (V. Vaidya, K. U. Ingold, D. A. Pratt, Angew. Chem. Int. Ed. 2009, 48, 157-160; P. T. Lynett, K. Butts, V. Vaidya, G. E. Garrett, D. A. Pratt, Org. Biomol. Chem. 2011, 9, 3320-3330). This is consistent with a previous report which provides a partition coefficient for allicin in phosphatidylcholine:water of Kp=11 (T. Miron, A. Rabinkov, D. Mirelman, M. Wilchek, L. Weiner, Biochim. Biophys. Acta 2000, 1463, 20-30). The Cope elimination of 3 from petivericin was roughly identical—with a half-life of ˜90 minutes (see Supp. Info.). While this Cope elimination is a reversible reaction in non-H-bonding organic solvents, (P. T. Lynett, K. Butts, V. Vaidya, G. E. Garrett, D. A. Pratt, Org. Biomol. Chem. 2011, 9, 3320-3330) it can be rendered irreversible by the reaction of either the sulfenic acid or the thiobenzaldehyde products that are formed. Without wishing to be bound by theory, it seems reasonable to suggest that the latter reacts rapidly with water at the lipid/aqueous interface, precluding the reverse reaction. Regardless, given that Cope elimination from both allicin and petivericin is still relatively facile in liposomes, the concentration of sulfenic acid formed must be too low at all times for it to compete with H₂B-PMHC for peroxyl radicals.

Hydrogen-bond accepting solvents are known to dramatically slow the rate of reaction between peroxyl radicals and sulfenic acids (e.g. 20-fold upon going from chlorobenzene to acetonitrile in the case of 7) (R. Amorati, P. T. Lynett, L. Valgimigli, D. A. Pratt, Chem. Eur. J. 2012, 18, 6370-6379), due to sequestration of the H-atom to be transferred as part of the H-bond (G. Litwinienko, K. U. Ingold, Acc. Chem. Res. 2007, 40, 222-230). Therefore, the lack of activity of allicin and petivericin in lipid bilayers could simply be the result of the sulfenic acids derived therefrom partitioning to the aqueous phase where they are much less reactive. To investigate this possibility, a lipophilic analog of 2 with alkyl chains appended to the para positions of the benzyl substituents was prepared (R. Amorati, P. T. Lynett, L. Valgimigli, D. A. Pratt, Chem. Eur. J. 2012, 18, 6370-6379) and investigated. It was thought that this modification would also make Cope elimination more reversible due to the greater lipophilicity of the thiobenzaldehyde product. However, oxidations of liposomes supplemented with 9 yielded fluorescence profiles that were essentially indistinguishable from those supplemented with allicin and petivericin.

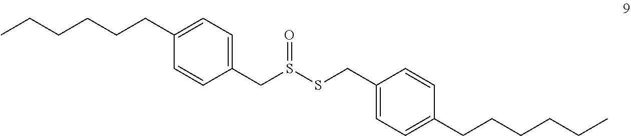 [SMILES: CCCCCCc1ccc(CSS(=O)Cc2ccc(CCCCCC)cc2)cc1]

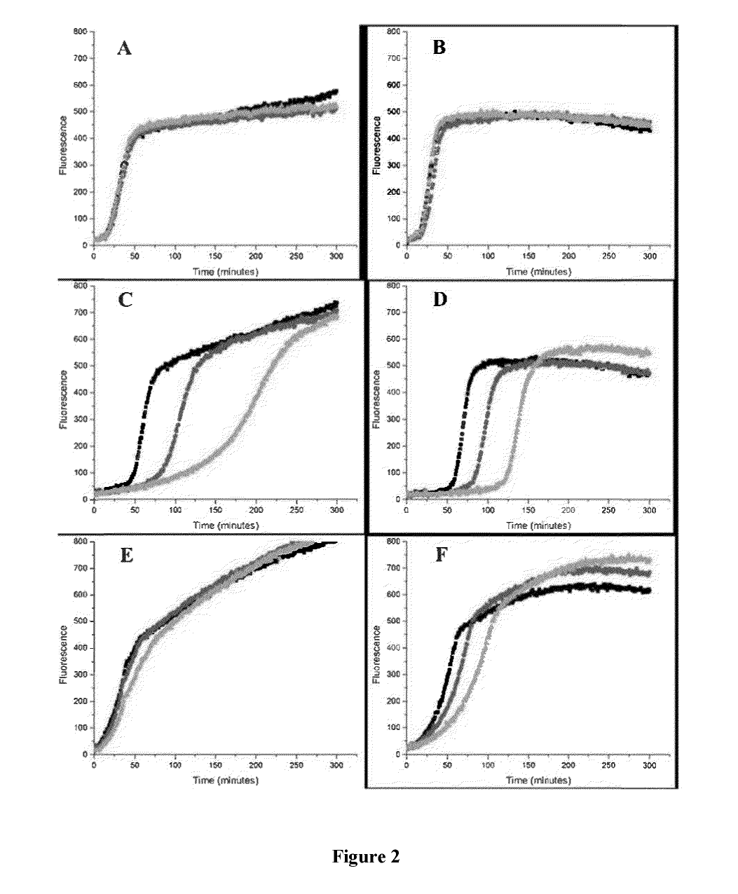

Cope elimination is not the only pathway by which sulfenic acids can form from thiosulfinates. In fact, sulfenic acids also arise from bimolecular reactions of thiosulfinates with nucleophiles, such as thiols (E. Block, Garlic and Other Alliums: the Lore and the Science, The Royal Society of Chemistry, Cambridge, UK, 2010; and E. Block, Angew. Chem. Int. Ed. Engl. 1992, 31, 1135-1178. Indeed, S-thiolation of thiosulfinates to form a sulfenic acid and a mixed disulfide (Eq. 3) is believed to be the first of two steps accounting for the observed stoichiometry in the reaction of a single molecule of thiosulfinate with two molecules of thiol to give 2 molecules of the mixed disulfide (Eq. 4). A possibility was that the addition of a water-soluble thiol, such as the ubiquitous cellular thiols glutathione or cysteine could promote sulfenic acid formation, thereby eliciting a strong radical-trapping activity. N-acetylcysteine (NAC) was selected as a model thiol and one equivalent was added to oxidations of liposome embedded H2B-PMHC supplemented with increasing amounts of allicin, petivericin or the lipophilic petivericin analog 9. Representative results are shown in FIG. 2, alongside profiles obtained when NAC alone is added to the buffer.

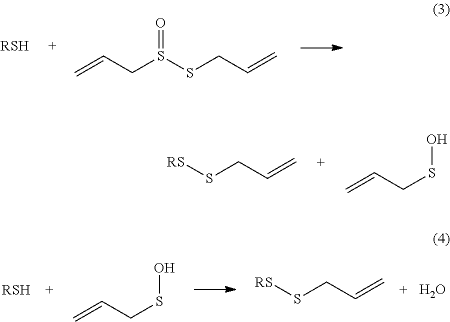

Under these conditions, 1 and 2 have little effect on the course of oxidations mediated by either the lipophilic or hydrophilic peroxyl radicals (FIGS. 2A and 2B). However, a pronounced inhibited period was observed in liposomes supplemented with 9 (FIGS. 2C and 2D). Since the presence of NAC alone does little in oxidations mediated by MeOAMVN (FIG. 2E) and has only a modest retarding effect in oxidations mediated by ABAP (FIG. 2F), this strongly suggests that NAC reacts with 9 to form a sulfenic acid that can partition to the lipid phase, where it reacts with peroxyl radicals, while the same reaction with 1 or 2 leads to a sulfenic acid that remains in the aqueous phase where it is consumed by reaction with another equivalent of NAC to yield mixed disulfides, which are expected to have no radical-trapping activity (R. Amorati, G. F. Pedulli, Org. Biomol. Chem. 2008, 6, 1103-1107). This is supported by the fact that the modest activity of NAC to inhibit the ABAP-mediated oxidation (FIG. 2F) disappears when allicin or petivericin are present (FIG. 2B and Supp. Info.)—and implies that the rate of the S-thiolation reaction is much faster than the rate of radical generation under these conditions. This is further supported by LC/MS measurements that were carried out to monitor the formation of the mixed disulfides arising from the reaction of either 2 or 9 with NAC over the course of the experiment, wherein it was observed that only half as much formed from 9 compared to 2.

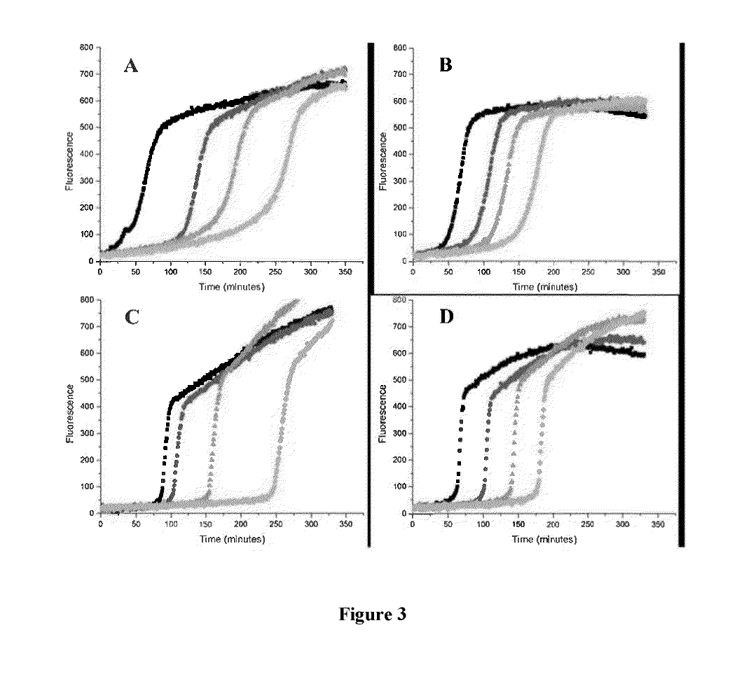

In the course of carrying out the foregoing experiments it was found that increasing the number of equivalents of NAC added to the aqueous phase proportionately increased the inhibited period observed in oxidations carried out in the presence of a given amount of 9. Representative results are shown in FIG. 3 for oxidations with both hydrophilic (FIG. 3A) and lipophilic peroxyl radicals (FIG. 3B). Since, in principle, only 1 eq. of NAC is necessary to produce the sulfenic acid by S-thiolation of 9, and the remaining NAC is not effective in inhibiting the oxidation (cf. FIGS. 2E and 2F), the result can only be explained by a synergistic interaction between NAC and the sulfenic acid derived from 9. To provide evidence for this synergism, analogous experiments were performed wherein a constant amount of the persistent sulfenic acid 5 was incorporated into the liposomes, and an increasing amount of NAC was added to the aqueous phase. Indeed, the same behaviour was observed (FIGS. 3C and 3D): the inhibited period was extended with increasing concentration of NAC, but the rate of inhibited oxidation remained the same, suggesting that the sulfenic acid was trapping the peroxyl radicals. It is noteworthy that the inhibited periods obtained for 9 in the presence of 1, 2, 3 and 5 equivalents of NAC correlate well with those obtained for 5 in the presence of 0, 1, 2 and 4 equivalents of NAC (recall that one equivalent of NAC must react with 9 to generate an equivalent of sulfenic acid in the first place).

The apparent interaction of NAC with the sulfenic acid derived from 9 or the persistent sulfenic acid 5 is reminiscent of the recycling of α-TOH by ascorbate (Eq. 5), which is believed to be key to the biological activities of both compounds in vivo (T. Doba, G. W. Burton, K. U. Ingold, Biochim. Biophys. Acta 1985, 835, 298-303; and B. Frei, L. England, B. N. Ames, Proc. Natl. Acad. Sci. USA 1989, 86, 6377). At the interface of the lipid bilayer and the surrounding aqueous medium, ascorbate can transfer an electron to the α-tocopheroxyl radical, either concerted with, or followed by, proton transfer to give α-TOH which can react with another peroxyl radical. However, in contrast to this process (which is ˜6 kcal/mol exothermic) (R. Amorati, G. F. Pedulli, L. Valgimigli, Org. Biomol. Chem. 2011, 9, 3792-3800), the regeneration of RSOH from H-atom transfer between RSO. and the thiol moiety of NAC (Eq. 6) is predicted to be highly endothermic (˜15 kcal/mol) (A. J. McGrath, G. E. Garrett, L. Valgimigli, D. A. Pratt, J. Am. Chem. Soc. 2010, 132, 16759-16761). Without wishing to be bound by theory, this suggests that an H-atom transfer process is unlikely, and instead, electron transfer from the thiolate to the sulfinyl radical seems more likely.

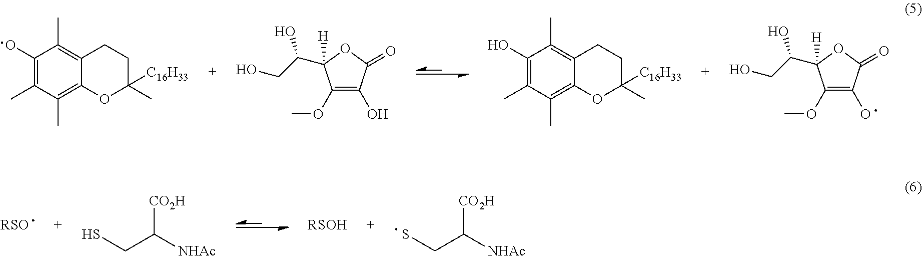

The results described above afford two key insights. First, they imply that allicin and petiviericin alone are not particularly effective as radical-trapping antioxidants in lipid bilayers, and that any antioxidant activity that is observed in vivo is likely to arise from other mechanisms (i.e., induction of antioxidant enzymes, for example, by depletion of glutathione by Eq. 3 and 4 or via reaction with oxidizable cysteines on signaling proteins). However, the combination of a lipophilic thiosulfinate and a water-soluble thiol is a potent co-antioxidant system in lipid bilayers, characterized by reactivity that is reminiscent of the cooperativity shown by Vitamins E and C. As well, the combination of a lipophilic sulfenic acid, such as 5, and a water-soluble thiol can form a potent co-antioxidant system in lipid bilayers.

### Example 4

### Synthesis and Characterization of S-2,4,6-Trimethylbenzyl Mesitylmethane-Sulfinothioate

S-2,4,6-trimethylbenzyl mesitylmethane-sulfinothioate was prepared according to the following scheme:

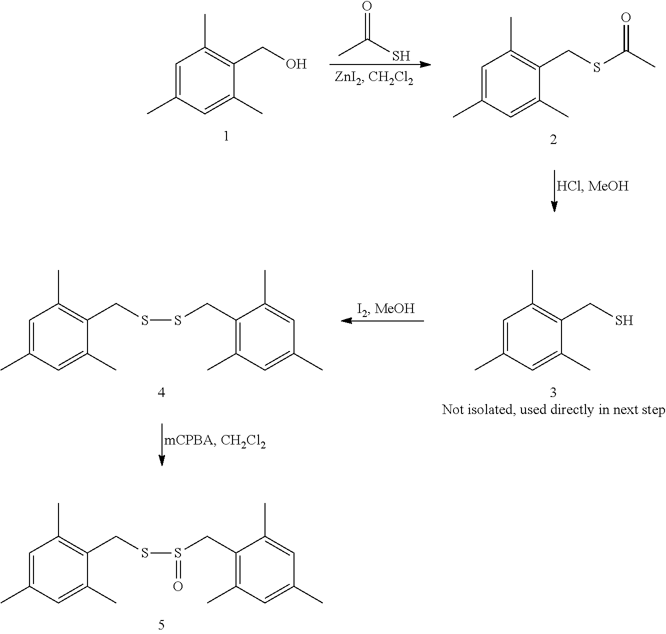

### S-2,4,6-trimethylbenzyl ethanethioate 2

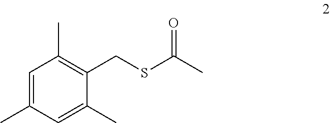

To a 2-necked 500 mL flask under nitrogen was added mesitylmethanol 1 (5 g, 33.3 mmol) to dichloromethane (133 mL, 0.25M) and all of the solid dissolved in the solvent. Zinc Iodide (5.31 g, 16.64 mmol) was added to the solution in one portion. The zinc iodide was not soluble in the solution, and white solid crashed out of the solution. Thioacetic acid (7.17 mL, 100 mmol) was added to the solution in one portion causing the solution to turn pale yellow. The flask was affixed with a water condenser and was covered in aluminum foil. The solution was heated to reflux for 17 hours and was allowed to cool to room temperature. Once cool, the solution was then quenched with water (40 mL) and this solution was transferred to a separatory funnel. The organic layer was collected and the aqueous layer was extracted with dichloromethane (4×7 mL). The combined organic fractions were washed with brine (1×100 mL), dried over sodium sulfate, filtered, and concentrated on the high vacuum to give the title compound 2 as a brown oil, 7.7 g, 111%.

¹H NMR (400 MHz, CDCl₃) δ 6.84 (s, 2H), 4.18 (s, 2H), 2.35 (s, 3H), 2.30 (s, 6H), 2.25 (s, 3H).

### Mesitylmethanethiol 3

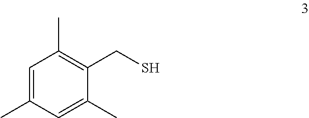

To a 2-necked 500 mL RBF containing a stir bar was added S-2,4,6-trimethylbenzyl ethanethioate 2 (5.5 g, 26.4 mmol) to methanol (191 mL, 0.138M) under nitrogen. Hydrochloric acid (2.3 mL, 26.4 mmol) was added slowly to the solution. The solution was affixed with a water condenser and was heated to reflux for 19 hours. An aliquot of the solution was removed, diluted with water, and extracted with methyl tert-butyl ether. The organic phase was concentrated on the high vacuum and was confirmed to be the title compound 3 by ¹H NMR. The solution was used directly in the next step.

1H NMR (400 MHz, CDCl₃) δ 6.48 (s, 2H), 3.74 (d, J=6.7 Hz, 2H), 2.36 (s, 6H), 2.25 (s, 3H).

### 1,2-bis(2,4,6-trimethylbenzyl)disulfane 4

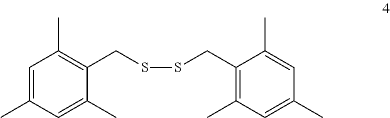

The 2-necked 500 mL RBF containing the mesitylmethanethiol 3 synthesized in the previous step was allowed to cool to room temperature, and was then cooled to 0° C. in an ice bath. Once cooled, a 5% iodine (3.69 g, 14.52 mmol) solution in methanol was added dropwise using an addition funnel over 30 minutes. During the addition solid crashed out of solution. The solution was allowed to stir for an additional 30 minutes in the ice bath. A 10% aqueous solution of sodium thiosulfate (100 mL) was added to quench the excess iodine. During the quench, more solid crashed out of solution. The solid was collected by vacuum filtration and was washed with freezer cold (−30° C.) methanol (3×50 mL). Residual solvent was removed from the solid on the high vacuum to yield the title compound 4 as an off white/light beige solid, 3.74 g, 86%.

¹H NMR (400 MHz, CDCl₃) δ 6.83 (s, 4H), 3.92 (s, 4H), 2.36 (s, 12H), 2.24 (s, 6H).

### S-2,4,6-trimethylbenzyl mesitylmethanesulfinothioate 5

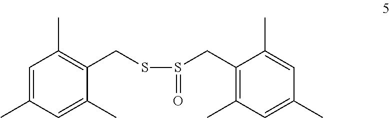

To a 2-necked 250 mL flask was added 1,2-bis(2,4,6-trimethylbenzyl)disulfane 4 (2.5 g, 7.56 mmol) to dichloromethane (32.4 mL, 0.19M) under nitrogen. The solution was cooled to 0° C. in an ice bath. Once cool, a solution of meta-chloroperoxybenzoic acid (2.8 g, 11.34 mmol) in dichloromethane (30 mL) was added dropwise using an addition funnel over 50 minutes. The solution was allowed to stir in the ice bath for an additional 30 minutes. To the cooled solution was added freezer cold (−30° C.) dichloromethane (70 mL). The solid that had crashed out of solution during the reaction was removed from the solution by filtering on a fritted funnel using vacuum filtration. The filter flask contained a saturated aqueous solution of sodium bicarbonate (100 mL) and a 10% aqueous solution of sodium sulfite (100 mL), and this quenched the reaction once it was filtered. The solution was transferred to a separatory funnel, and the two layers were separated. The aqueous layer was extracted with dichloromethane (2×30 mL), and the combined organic layers were then washed with water (1×50 mL), and brine (2×50 mL). The washed solution was dried over sodium sulfate, filtered and concentrated on the rotary evaporator to give a light yellow solid. The yellow solid was purified by column chromatography eluting from hexanes:ethyl acetate (20:1) to yield the title compound 5 as a white solid (650 mg, 25%).

R_{f} of title compound (hexanes:ethyl acetate, 9:1): 0.30; ¹H NMR (400 MHz, CDCl₃) δ 6.87 (s, 2H), 6.84 (s, 2H), 4.57 (d, J_{AB}=13.3 Hz, 1H), 4.44 (d, J_{AB}=13.3 Hz, 1H), 4.38 (d, J_{AB}=11.8 Hz, 1H), 4.33 (d, J_{AB}=11.8 Hz, 1H), 2.35 (s, 6H), 2.32 (s, 6H), 2.26 (s, 3H), 2.25 (s, 3H); ¹³C NMR (400 MHz, CDCl₃) δ 138.27, 138.24, 137.69, 137.42, 129.57, 129.35, 129.20, 125.16, 57.62, 31.69, 21.14, 21.10, 20.90, 19.86; HRMS (ESI) calcd for C₂₀H₂₆OS₂[Na]⁺ 369.1323. found 369.13182.

### Example 5

### Synthesis and Characterization of S-(Furan-2-Ylmethyl) Furan-2-Ylmethane-Sulfinothioate

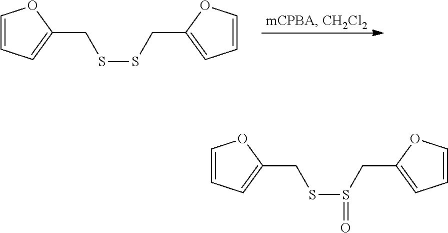

To a 500 mL round bottomed-flask was placed furfuryl disulfide (5.00 g, 22.1 mmol) followed by 80 mL of DCM. The resulting solution was cooled to 0° C. in an ice-water bath. The flask was fitted with a dropping funnel that was charged with a solution of m-CPBA (5.45 g, 77% titer, 24.3 mmol) in DCM (65 mL). The m-CPBA solution was added dropwise to the reaction mixture over the course of 30 min, and a white precipitate formed. The mixture was allowed to stir for a further 15 minutes at 0° C. The precipitate was removed by filtration through a scintered glass funnel using vacuum filtration, and the filtrate was quenched by addition of saturated aqueous NaHCO₃ (100 mL) and 10% aqueous NaSO₃ (50 mL). The organic layer was separated and the aqueous was extracted with one portion of DCM (25 mL). The combined organic layers were washed with brine (20 mL) and then dried over Na₂SO₄. Evaporation of the solvent under reduced pressure gave viscous red oil that was purified by column chromatography (10% EtOAc/Hexanes eluent). This gave the title compound as a red oil (0.260 g, 5% yield).

R_{f} of title compound (hexanes:ethyl acetate, 9:1): 0.15; ¹H NMR (400 MHz, CDCl₃) δ 7.44 (dd, J=1.9, 0.8 Hz, 1H), 7.38 (dd, J=1.9, 0.8 Hz, 1H), 6.44 (dd, J=3.3, 0.8 Hz, 1H), 6.39 (dd, J=3.3, 1.9 Hz, 1H), 6.32 (dd, J=3.3, 1.9 Hz, 1H), 6.30 (dd, J=3.3, 0.8 Hz, 1H), 4.43 (d, J_{AB}=14.0 Hz, 1H), 4.38 (d, J_{AB}=11.0 Hz, 1H), 4.34 (d, J_{AB}=12.0 Hz, 1H), 4.29 (d, J_{AB}=14.9 Hz, 1H); ¹³C NMR (400 MHz, CDCl₃) δ149.70, 144.15, 143.90, 142.99, 112.04, 111.30, 110.86, 109.11, 54.98, 28.15; HRMS (ESI) calcd for C₁₀H₁₀O₃S₂[H]⁺ 243.01496. found 243.01439.

### Example 6

### Kinetics of Reaction of S-(Furan-2-Ylmethyl) Furan-2-Ylmethanesulfinothioate with Peroxyl Radicals

The peroxyl radical clock method was used to measure the rate constants for the reaction of a furan-containing thiosulfinate compound (S-(furan-2-ylmethyl) furan-2-ylmethanesulfinothioate) with peroxyl radicals (kᵢₙₕ). The method relies on the competition between the β-fragmentation of a peroxyl radical and its trapping by a H-atom donor. When methyl linoleate is used as a precursor, the reduction of the two initially formed (Z,E)-dienylperoxyl radicals by the antioxidant (A-H in the scheme below) competes with their isomerization to the more thermodynamically stable (E,E)-dienylperoxyl radicals. Therefore, the ratio of (Z,E) to (E,E) diene hydroperoxides that were formed as a function of [A-H] was used to determine the second order rate constant (kᵢₙₕ) for the reaction of the antioxidant with the dienylperoxyl radicals.

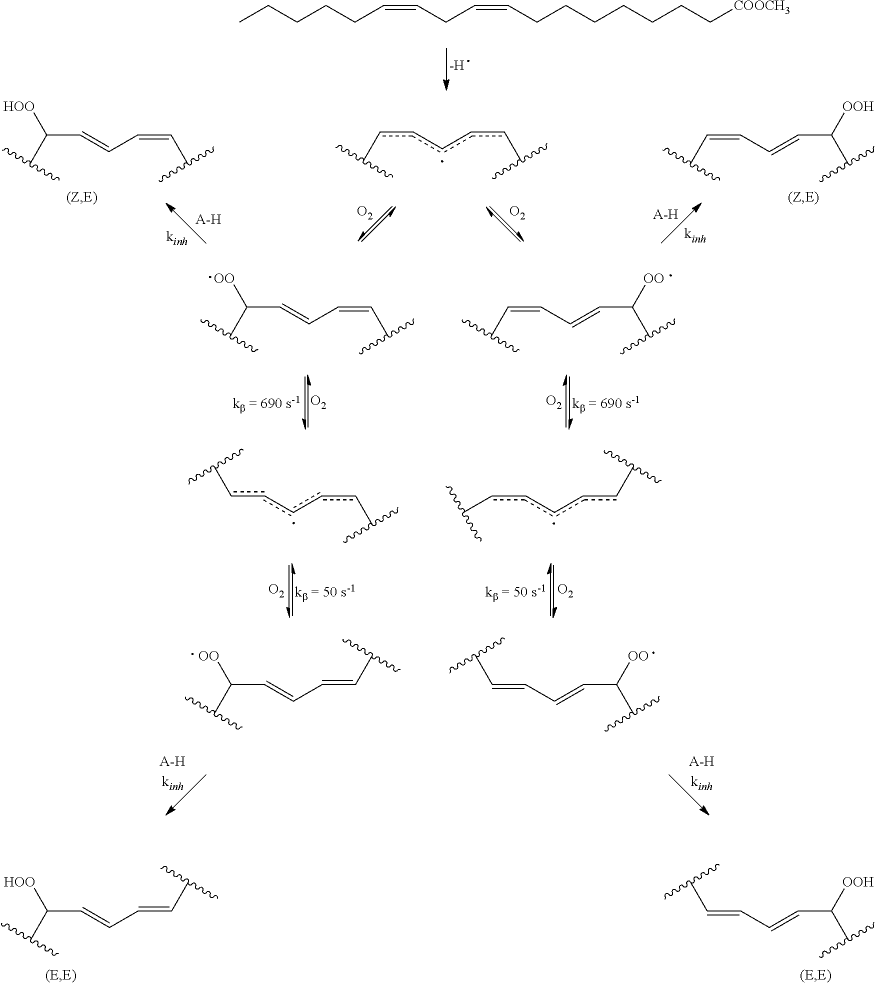

The results show that the furan-containing thiosulfinate compound had antioxidant activity similar to that of p-n-C₆H₁₃-BPT 5 (see Example 2 above).

TABLE 1
Inhibition Rate Constants for Reactions of
Thiosulfinates with Peroxyl Radicals in
Chlorobenzene at 37° C. Using the
Peroxyl Radical Clock Approach

| | kᵢₙₕ/kᵢₙₕ |
|---|---|
| Thiosulfinate | (C6BPT) |
| 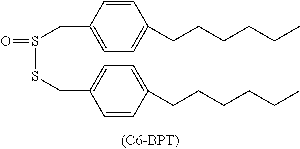 [SMILES: CCCCCCc1ccc(CSS(=O)Cc2ccc(CCCCCC)cc2)cc1] | 1.0^{a} |
| 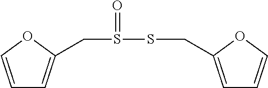 [SMILES: O=S(Cc1ccco1)SCc1ccco1] | 1.1 |

^{a}The value of kᵢₙₕ for C6BPT was previously determined to be 4 × 10⁴ M⁻¹s⁻¹.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

The embodiments of the invention in which an exclusive property or privilege is claimed are defined as follows:

1. A compound of Formula X 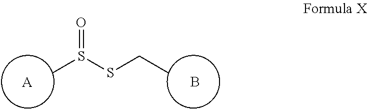
wherein
A is an optionally substituted aliphatic or an optionally substituted aryl, and
B is a moiety that stabilizes the partial positive charge at the carbon adjacent the divalent sulfur,
wherein the compound is not allicin, S-benzyl phenylmethane, petivericin, or methylpetivericin.

2. The compound of claim 1, wherein A and B, together with the rest of the compound, form a cycle.

3. The compound of claim 1, wherein B is a substituted olefin, or an optionally substituted aryl.

4. The compound of claim 3, having the structure of Formula I 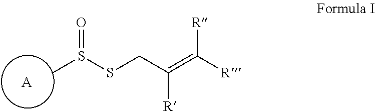
wherein
R′ is hydrogen, or optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester, and
R″ and R′″ are each independently optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester,
wherein any two or more of R′, R″ and R′″ together with the carbon atoms to which they are attached form an optionally substituted cycloalkyl or aryl ring.

5. The compound of claim 4, wherein R′ is hydrogen, and R″ and R′″ are each independently an optionally substituted alkyl or an optionally substituted aryl.

6. The compound of claim 4, wherein R′ and R″ together with the carbon atoms to which they are attached form an optionally substituted aryl ring, such as a phenyl, a naphthyl, an anthracenyl, a tripticenyl, a furanyl, a pyridinyl, pyrrole, thiophenyl.

7. The compound of any one of claim 1, 3, 4, 5 or 6, wherein A is —CH₂-aryl, which is optionally substituted.

8. The compound of claim 6, having the structure

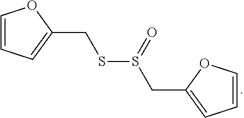 [SMILES: O=S(Cc1ccco1)SCc1ccco1]

9. The compound of claim 1, wherein the compound has structure of Formula Ia 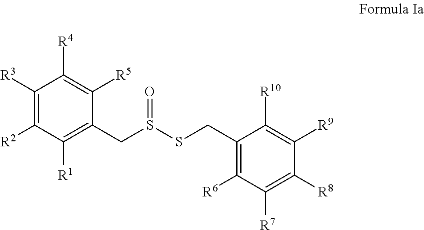
where R¹, R², R³, R⁴, R⁵, R⁶, R⁷, R⁸, R⁹ and R¹⁰ are each independently H or an optionally substituted alkyl, optionally substituted alkenyl, optionally substituted alkynyl, optionally substituted aryl, optionally substituted heteroaryl, optionally substituted cycloalkyl, Si(alkyl)₃, Si(alkoxy)₃, halo, alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, nitro, nitrile, azido, heterocyclyl, ether, or ester,
wherein at least one of R¹, R², R³, R⁴, R⁵, R⁶, R⁷, R⁸, R⁹ and R¹⁰ is not H, and
wherein when only one of R¹, R², R³, R⁴, R⁵, R⁶, R⁷, R⁸, R⁹ and R¹⁰ is not hydrogen, then one of R¹, R², R³, R⁴, R⁵, R⁶, R⁷, R⁸, R⁹ and R¹⁰ is other than methyl.

10. The compound of claim 9, wherein at least one of R¹, R², R³, R⁴ and R⁵ is not H and at least one of R⁶, R⁷, R⁸, R⁹ and R¹⁰ is not H.

11. The compound of claim 9, wherein R¹, R⁵, R⁶ and R¹⁰ are H.

12. The compound of claim 9, wherein R³ and R⁸ are each independently an optionally substituted C₄-C₂₀ alkyl.

13. The compound of any one of claims 9-12, wherein R³ and R⁸ are the same.

14. The compound of claim 9, having the structure:

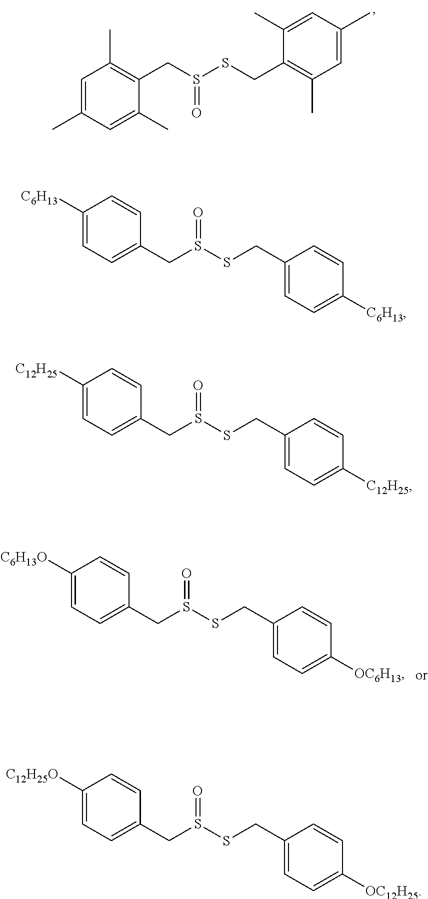

15. The compound of any one of claims 1-14 for use as an anti-oxidant.

16. A composition comprising a lipophilic sulfenic acid and a water soluble thiol.

17. The composition of claim 16, wherein the water soluble thiol is glutathione, cysteine, or N-acetyl cysteine.

18. A composition comprising a compound of formula X 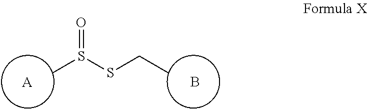
and a water soluble thiol, wherein
A is a lipophilic moiety, and
B is a moiety that stabilizes the partial positive charge at the carbon adjacent the divalent sulfur.

19. The compound of claim 18, wherein A is a C₁₀-C₃₀ optionally substituted aliphatic or a C₁₀-C₃₀ optionally substituted aryl.

20. The compound of claim 18 or 19, wherein B is a substituted olefin, or an optionally substituted aryl.

21. The compound of any one of claims 18-20, wherein A is —CH₂-aryl.

22. A method of inhibiting oxidation of a molecule comprising the step of mixing the molecule with a compound of any one of claims 1-14.

23. An antioxidant composition comprising a compound of any one of claims 1-14 and, optionally, a diluent or excipient.

24. A stabilized composition comprising a compound of any one of claims 1-14 and a molecule or mixture requiring protection from radical oxidation.

25. A process for preparing the compound of Formula Ia according to claim 9, comprising the steps of:
a) reacting a compound of Formula II: 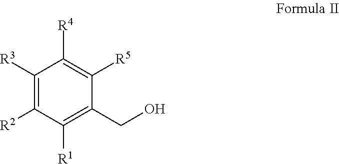
wherein R¹, R², R³, R⁴, and R⁵ are as defined in claim 9,
with thioacetic acid and zinc iodide to form the compound of Formula III: 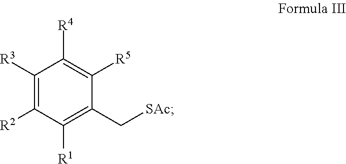
b) converting the compound of Formula III to the thiol of Formula IV: 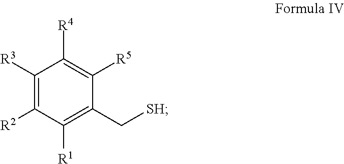
c) oxidizing the compound of Formula IV to form the disulfide of Formula V: 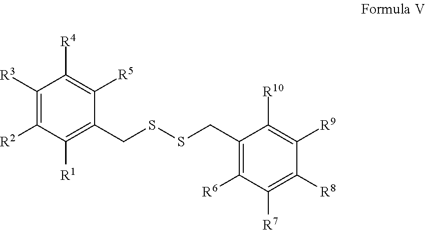
wherein R⁶, R⁷, R⁸, R⁹, and R¹⁰ are as defined in claim 9; and
d) further oxidizing the disulfide of Formula V to form the compound of Formula Ia:

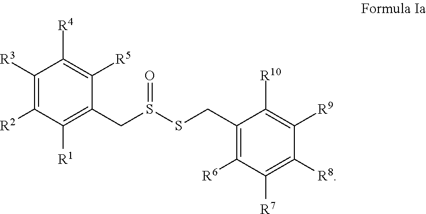

26. Use of the composition of any one of claims 18-21 for inhibiting oxidation of a molecule in a lipid bilayer or a lipoprotein.

27. The compound of any one of claims 1-14 for use in stabilizing gasoline, crude oil, or petroleum fuels.
